(19) United States
(12) Patent Application Publication
(10) Pub. No.: US 2014/0148782 A1
(43) Pub. Date: May 29, 2014

(54) TISSUE INFUSION SYSTEM AND METHOD

(71) Applicants: Rick M. Odland, Roseville, MN (US); Bradford G. Staehle, Minnetonka, MN (US); Scott R. Wilson, Maple Grove, MN (US); Michael R. Wilson, Minneapolis, MN (US)

(72) Inventors: Rick M. Odland, Roseville, MN (US); Bradford G. Staehle, Minnetonka, MN (US); Scott R. Wilson, Maple Grove, MN (US); Michael R. Wilson, Minneapolis, MN (US)

(73) Assignee: TWIN STAR MEDICAL, INC., Minneapolis, MN (US)

(21) Appl. No.: 13/698,485

(22) PCT Filed: May 27, 2011
(86) PCT No.: PCT/US11/38394

§ 371 Date: Jun. 18, 2013

Related U.S. Application Data
(60) Provisional application No. 61/349,595, filed on May 28, 2010.

(51) Int. Cl. A61M 25/00; A61M 25/04; A61B 18/06; A61M 25/01

(52) U.S. Cl. CPC A61M 25/0068; A61M 25/0043; A61M 25/0105; A61M 25/04; A61B 18/06
USPC 604/506; 604/246; 604/174; 604/170.02; 604/164.04

(57) ABSTRACT

An apparatus and corresponding method for providing convection enhanced delivery of bioactive agent to a tissue site. The apparatus involves the use of semipermeable membranes, typically in the form of one or more hollow fibers, together with a source and conduit of solution containing the bioactive agent to be delivered. The use of hollow fiber technology provides an optimal combination of features, including delivery kinetics and distribution, as compared to conventional (e.g., standard needle) type delivery devices.

### TECHNICAL FIELD

In one aspect, the present invention relates to systems and apparatuses for tissue infusion, e.g., by convection enhanced delivery (CED). In another aspect, the invention relates to catheters that include semipermeable membranes, in the form of hollow fibers, for use in delivering or recovering materials to or from the body.

### BACKGROUND OF THE INVENTION

A variety of bioactive agents have been described for use in treating parts of the body, often by direct, localized injection to the body part itself. Typically, such injections are sufficient to deliver the necessary dose to the body, but in many instances, conventional delivery of this type suffers from various drawbacks, including with respect to the difficulty in accessing various parts of the body and/or the ability to achieve effective or desired delivery kinetics and/or distribution.

For instance, Benign Prostatic Hyperplasia (BPH) will become an increasing burden on economic resources with the aging population. Surgical treatment is well established and has provided satisfactory results in 60-80% of men. However, it has been associated with significant morbidity and complication, therefore significant efforts have been directed toward developing alternative minimally invasive treatments. Ablation of the prostate by direct injection has the potential to significantly reduce expense and morbidity; drugs are available to chemically ablate the tissue. However, though direct injection would appear to be a straightforward approach to the problem, backflow along the needle track and uneven distribution of drug after injection are significant drawbacks to chemoablation.

See Applicant's own US Patent Publication Nos. US-2005-0165342, US-2010-0106140, US-2010-0100061, and U.S. Pat. Nos. 6,030,358, 6,537,241, 6,942,633, 6,942,634, and 7,717,871, the disclosures of which are incorporated herein by reference.

### BRIEF DESCRIPTION OF THE DRAWINGS

FIG. 1 illustrates the expected results of distribution of dye with a hollow fiber catheter and a standard injection needle.

FIG. 2 depicts a schematic demonstrating options for interstitial flow and impedance mismatch.

FIGS. 3A-3C illustrate the procedure of placement of a hollow fiber into the prostate.

FIGS. 4A-4B provide cross sectional views of a preferred infusion catheter according to the current invention.

FIGS. 5A-5B provide a cross sectional view and a top view of a preferred needle retraction device according to the current invention.

FIGS. 5C-5D provide a top view and a cross sectional view of a preferred needle retraction device according to the current invention.

FIGS. 6A-6E provide cross sectional views illustrating an optional catheter priming method according to the current invention.

FIGS. 7A-7B show cross sectional views of a preferred distal tip anchor mechanism according to the invention.

FIGS. 8A-8B show cross sectional views of a preferred proximal anchor mechanism according to the invention.

FIGS. 9A-9B show cross sectional views of a preferred mechanism for providing suction according to the invention.

FIGS. 10A-10B show alternative embodiments of a catheter having a priming lumen.

FIG. 11 provides a cross sectional view of a proximal end of a catheter with a priming lumen.

FIG. 12 depicts a diagram describing the flow of priming fluid using a catheter having a priming lumen.

FIG. 13 shows a side cross-sectional view of the distal end of a catheter having a priming lumen.

FIGS. 14A-14C provide side and cross sectional views of a further preferred needle retraction device according to the current invention.

FIGS. 15A-15D provide views of an alternative needle retraction device, including the use of a curved catheter tip.

### SUMMARY OF THE INVENTION

The present invention provides a system, including an apparatus and corresponding method for the direct infusion of bioactive agents into a bodily tissue site, by means of convection enhanced delivery. The apparatus is adapted to provide an optimal combination of properties, including clinically relevant infusion rate and distribution, as well as ease of operation, while also avoiding or minimizing undesired properties such as backflow, irregular distribution patterns, undesired tissue shear, etc. These and other results can be obtained or improved by a variety of means, including anchoring means to retain the catheter in the desired tissue site during infusion, and/or by priming means, to ensure that delivery is optimized without the appearance of undue obstruction by air and in a manner that permits controlled delivery of desired infusate (material to be infused into the tissue site) to the tissue site.

In a preferred embodiment, the apparatus comprises:

a) one or more hollow fiber catheters adapted to be placed and retained in a desired position within a tissue site, and following use removed from the tissue, without undue damage to the tissue;

b) a bioactive agent circuit adapted to deliver bioactive agent from a source and to the catheter(s) in order to be delivered to the tissue site; and

c) a control mechanism adapted to permit the controlled delivery of bioactive agent to the catheter(s) in a predetermined manner to the tissue site.

In a further preferred embodiment the one or more hollow fiber catheters are adapted to be placed into and positioned within the tissue site using ancillary means, e.g., a removable sheath, trocar and/or stylet that provides suitable properties to the catheter in the course of its delivery, e.g., strength, rigidity, and the ability to be steered or otherwise positioned, as well as the ability to be tracked or located by suitable means.

In another preferred embodiment, the one or more catheters are adapted to be substantially retained in position within the tissue site during the course of infusion, e.g., by the use of distal and/or proximal anchor mechanisms.

In a further preferred embodiment, the bioactive agent circuit is adapted to deliver bioactive agent substantially without the presence of air or other occlusions, e.g., by means of first priming the catheter with a suitable solution, in order to provide for better and more accurate delivery.

In another preferred embodiment, the control mechanism permits one handed operation of the apparatus and/or one or more delivery controls, e.g., with respect to control of position, flow rate, timing (e.g., continuous, periodic, intermittent delivery), and corresponding detectors (e.g., occlusion detection). In yet another preferred embodiment, the bioactive agent is delivered by any suitable means, e.g., by the application of hydrostatic pressure, the use of convective or osmolar forces, and the like.

A method of this invention provides:

a) providing an apparatus as described herein,

b) placing and retaining the catheter(s) within a tissue site, in a manner that positions the hollow fiber portion in an desired position and orientation with respect to the tissue,

c) activating the apparatus in order to deliver bioactive agent to the tissue in a manner that provides an optimal and predetermined combination of properties selected from the group consisting of delivery kinetics (e.g., flow rate, total amount) and distribution (e.g., homogeneous, symmetric, predetermined configuration, minimal reflux or backflow).

### DETAILED DESCRIPTION

Applicant has discovered that by the use of hollow fiber catheters, as compared to conventional needle tips, bioactive agents can be delivered by means of convection enhanced delivery in a manner that can be better controlled, and in turn, as or more effective than conventional tips, while providing fewer corresponding risks or drawbacks. This is particularly the case where, as presently provided, the delivery catheter can be suitably positioned and retained in the tissue site, and in turn, delivery of the bioactive agent can be facilitated by first priming the catheter, e.g., with a suitable amount and type of solution.

Those skilled in the art, given the present description, will appreciate the manner in which the apparatus and method can be adapted for use with various bioactive agents and corresponding solutions containing such agents, as well as the physiologic parameters of various patients and tissue sites, in order to deliver bioactive agent in a controlled and desired manner.

Those skilled in the art, given the present disclosure, will appreciate the manner in which the extent of infusate distribution that is achieved using CED can depend on many factors, including factors associated with the patient (e.g., hydraulic conductance of the tissue, interstitial pressure, type of tissue infused), with the bioactive agent solution itself (e.g., infusate concentration, molecular weight, osmolarity, hydrophobicity, etc.), and with the apparatus used to deliver the solution (e.g., volume and flow rate during administration, as well as position, diameter, porosity, and type of administration catheter(s)). As used herein, the term “bioactive agent” will refer to any infusate that is delivered to the tissue site in order to provide a desired biological and/or physical-chemical response. In turn, the bioactive agent can include an infusate where the active agent is provided in the form of a homogeneous solution, emulsion, or other suitable form. Examples of suitable infusates include, for instance, solutions of bioactive drugs or other pharmaceuticals, as well as solutions that themselves provide a physical-chemical response, such as ablation by the delivery of ethanol or other suitable agents.

Direct infusion into the tissue has been studied in many tissues, each of which tends to present unique challenges. For instance, conventional CED has been shown to be useful for delivery of agents to the brain; however, to avoid problems of backflow and shearing, the rate of infusion must be very slow. The apparatus and method of the present invention involves the application of hollow fiber technology to further enhance CED. In contrast to conventional drug delivery catheters, the hollow fiber delivery catheter is sufficiently (e.g., completely) porous to the flow of infusate solution and/or its components, thereby considerably increasing the surface area of tissue that can be contacted with infusate. In one preferred embodiment, for instance, a hollow fiber having millions of micropores that average only 0.45 μm in diameter, can be used to create multiple pathways for outflow of bioactive agent into the tissue. In turn, Applicant has demonstrated the manner in which the use of such hollow fibers can minimize or eliminate unwanted properties, such as shear plane and reflux, in the course of bioactive agent delivery, and in turn, increase the amount of bioactive agent delivered to the site, as well as the desired localization of agent within the site.

The method and apparatus of the present invention can be used to deliver a continuous and/or periodic (e.g., intermittent) infusion of bioactive agents such as drugs via indwelling catheters, thereby enabling convective distribution of bioactive agent in desired concentrations and over desired volumes of the target tissue, while minimizing or avoiding systemic toxicity.

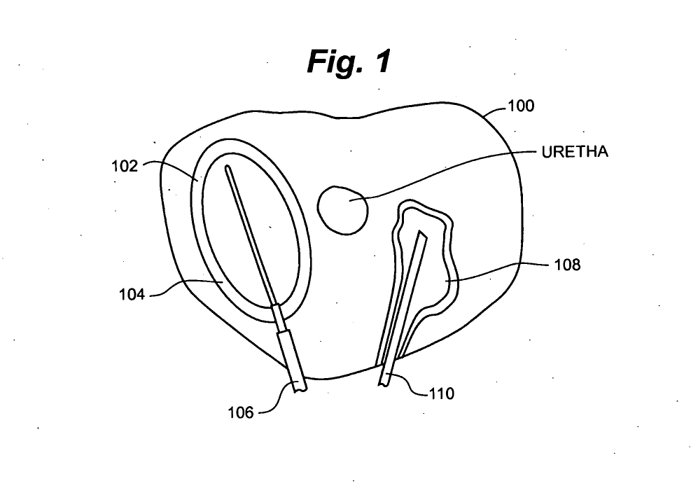

Turning to the drawings, FIG. 1 illustrates expected patterns of dye distribution within a prostate capsule 100. On the left side of the prostate capsule 100, an example dye distribution 102 resulting from a hollow fiber catheter 104 and guide needle 106 combination is shown. On the right side of the prostate capsule 100, an expected dye distribution 108 resulting from a standard single end port injection needle 110 is shown. Some embodiments of the invention are directed to eliminating, or at least reducing, the backflow and asymmetric distribution 108 associated with standard injection needle techniques.

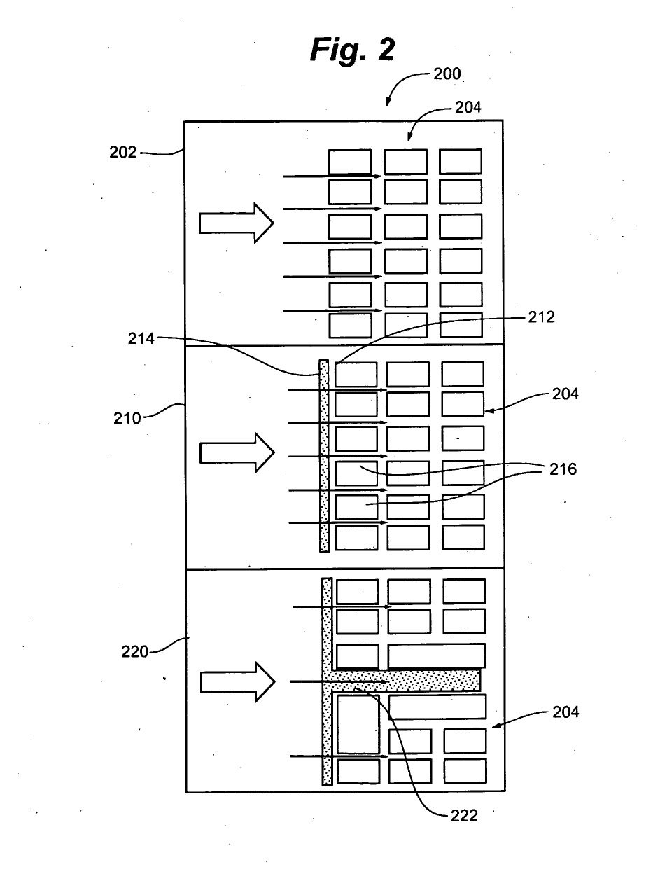

FIG. 2 depicts a schematic 200 illustrating interstitial flow and impedance mismatch that can occur when flow into the tissue by infusion exceeds the capacity of flow within the tissue. In an idealized flow situation 202, every cell in the tissue 204 is perfused with convective fluid flow. In a setting 210 in which the infusion rate is increased, an impedance mismatch exists, and all the fluid delivered by the catheter does not flow into the tissue 204. The resultant increase in pressure at the fluid-tissue interface 212 creates force (illustrated by bar 214) that deforms the tissue 204, which in turn decreases the size of the interstitial pathways 216 nearest the catheter, and further increases resistance to tissue flow. In a situation 220 with continued infusion, rising pressure can create a rupture 222 in the tissue 204. Any therapeutic agent in this interstitial pool can then only reach the cells by diffusion.

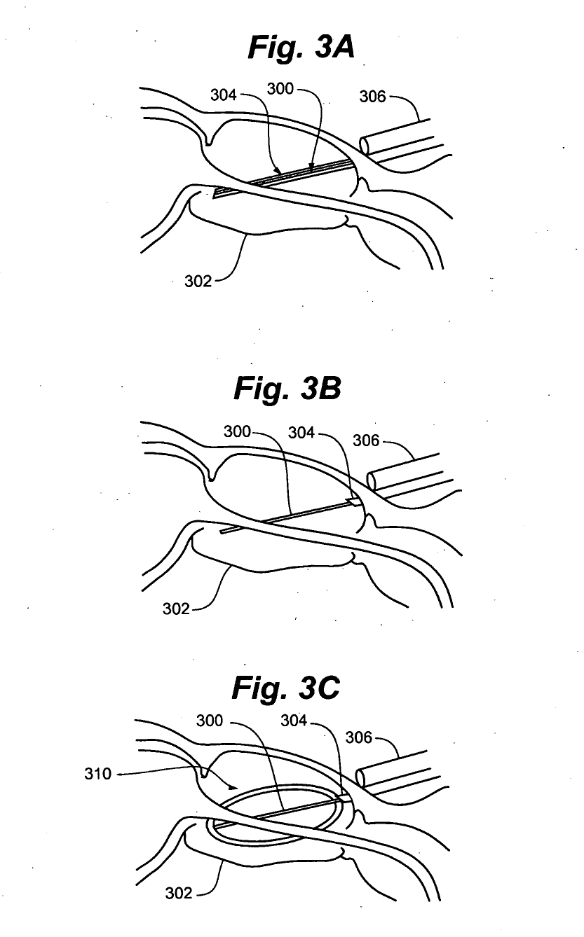

FIGS. 3A-3C illustrate an example of a procedure for placing and positioning a hollow fiber catheter 300 into the prostate 302. A guide needle cannula 304 will be placed transrectally into the prostate gland 302. Preferably, the hollow fiber catheter 300 will first be primed and then placed into the needle 304 (e.g., before or after needle placement within the prostate) such that the distal tip is just behind the needle point. The needle and catheter combination can be inserted into the prostate 302 to the desired location as confirmed, for instance, using a transrectal ultrasound (TRUS) probe 306. The needle 304 will be withdrawn, leaving the exposed hollow fiber catheter 300 in the desired location for infusion of therapeutics. The injectate will be infused through the hollow fiber 300 to create a well distribution of material 310 throughout the prostate 302.

Upon completion of the injection, the hollow fiber catheter 300 is preferably removed by simply pulling on the catheter shaft. Applicant has used the same catheter design in human muscle compartments, and found that as long as there are substantially no raised edges along the length of the catheter in contact with tissue, the hollow fiber catheters can be removed without complications, even through muscle fascia and skin.

Alternatively, the catheter 300 can be placed with other imaging modalities and methods. For instance, intraoperative MR, CT and fluoroscopy are the additional imaging modalities that can be used real time to see catheter position in relation to targeted tissue. In addition, treatment planning software can be used with pre-placement images that include reference markers to indicate location of catheters in relation to the targeted tissue as provided by pre-catheter placement image. In some instances, catheter guides separate from the imaging device are used to guide the catheter to the correct position. Finally, treatment planning software can be used to determine the most desirable catheter placement providing the number and location of catheters and then the preferred infusion protocol, including flow rate and volume.

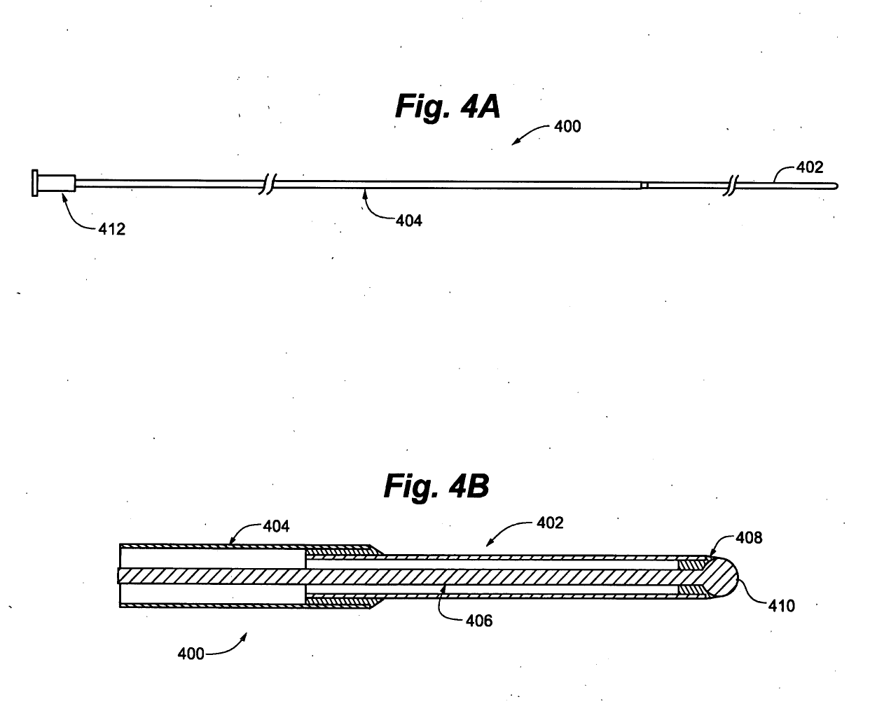

FIGS. 4A-4B provide cross sectional views of a preferred infusion catheter 400 according to the current invention. As shown, the catheter 400 includes a 1-3 cm hollow fiber 402 (e.g., 0.45 Micron, 0.28×0.36 mm) bonded to an extra-thin wall stainless steel or coil-reinforced polyimide tube 404. A 0.006″ stainless steel stylet 406 is bonded to the distal end 408 of the fiber to provide mechanical strength. The distal tip of the stylet 406 includes a ball 410 to strengthen the bond joint. A luer fitting 412 is provided at the proximal end of the catheter 400 to allow connection to a fluid line.

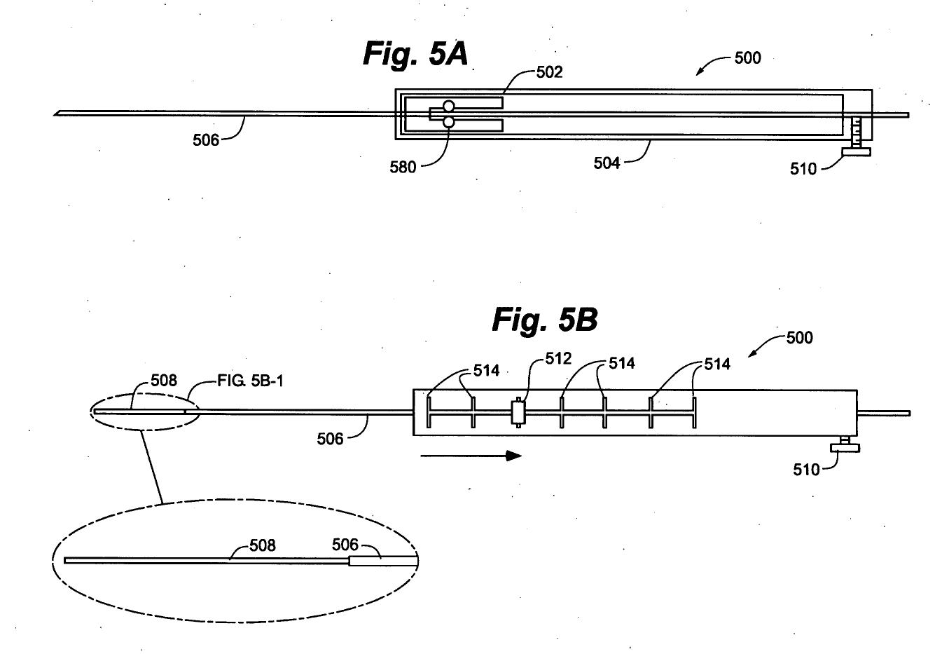

FIGS. 5A-5B provide a cross sectional view and a top view, respectively, of a preferred needle retraction device 500 according to the current invention. (FIGS. 5C-D, 14A-C, and 15A-D provide further examples of retraction devices.) In order to facilitate ease of needle retraction, the device 500 may allow for one handed operation. The device 500 includes an inner adjustable body 502 positioned within an outer body 504 adapted to be grasped by the hand. The inner adjustable body 502 is connected with needle 506, and allows for sliding the needle 506 proximally and distally with respect to the outer body 504 and hollow fiber catheter 508 fixed relative to the outer body with locking screw 510. Exterior to the outer body 504, a sliding handle 512 can be moved between two or more position locks 514 to adjust the position of the inner adjustable body 502 and needle 506.

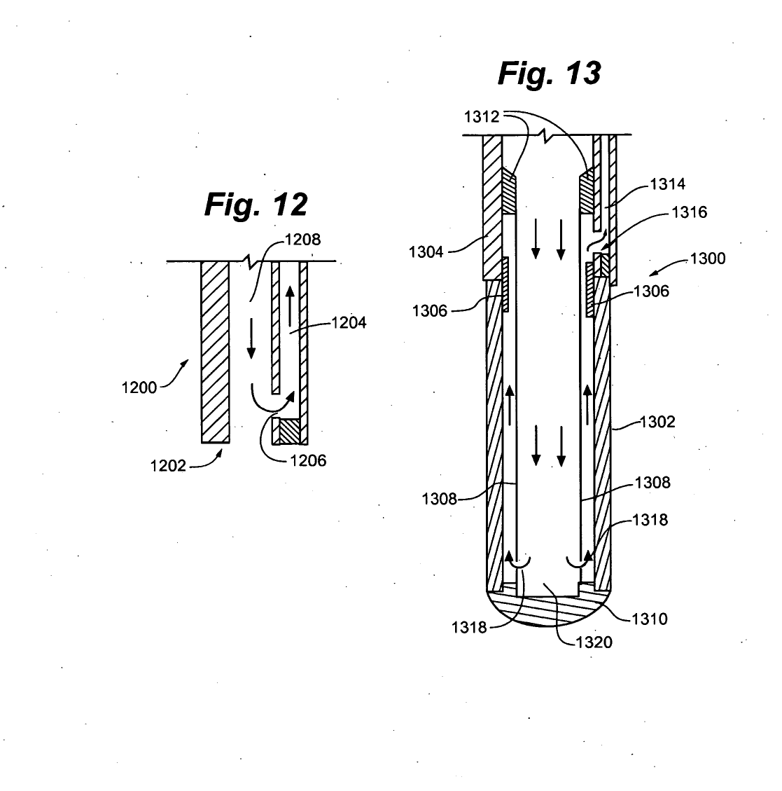

FIGS. 5C-5D provide a top view and a cross sectional view, respectively, of a preferred needle retraction device 550 with a spring mechanism 570 to provide the force for the retraction of the needle 556 according to the current invention. The device 550 includes of a spring-loaded inner shuttle 502 used to retract the needle 556 and expose the hollow fiber catheter 558. First, the catheter 558 (e.g., any of the catheters described herein, including the example of FIG. 13) is loaded and secured to the handle 504 with part A and locking screw 560. Next, the shuttle 552 (part B) is pushed forward to compress the spring 570 and allow the needle 556 to cover the fiber 558 for insertion. Once the needle 556 is placed into the tissue, a button 572 is pushed and the needle 556 retracts into an outer sheath 574 attached to the handle 554. The spring 570 should have sufficient force to overcome tissue and o-ring resistance. The outer sheath 574 helps with maintaining catheter position in tissue. Two o-rings 580 are placed within the handle 554 to provide air-tight seals and prevent fluid backflow up through the needle and the sheath. A luer fitting (not shown) will be attached to part A for easy fluid line connection.

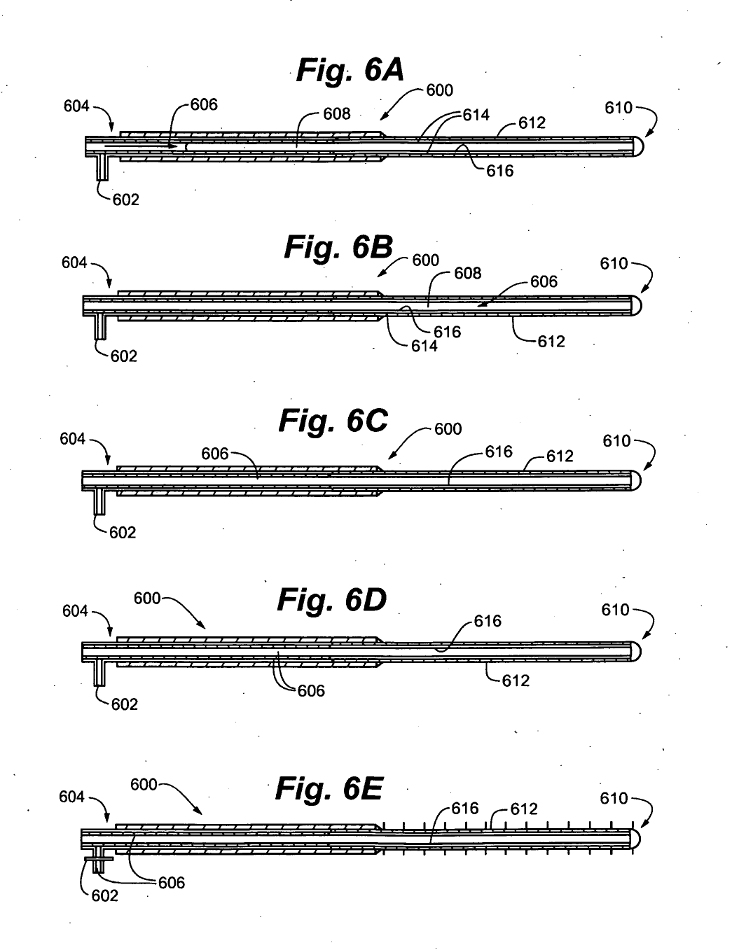

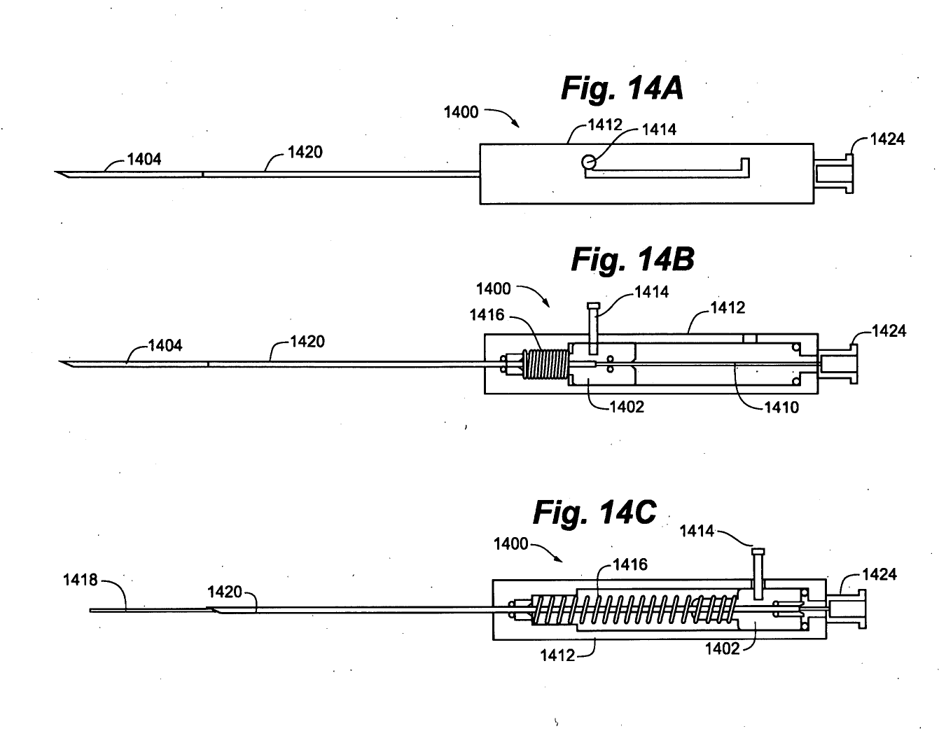

Generally, the catheters 508, 558 shown in FIGS. 5A-5D are primed prior to placement in the tissue. However, in some instances this may not be practical, such as with the need for a removale stylet or even in some applications with a fixed stylet. There may be instances when one removes the catheter of FIG. 4 from the needle and associated retraction device found in FIGS. 5A-5B, 5C-5D, 14A-14C, and/or 15A-15D. This may be desirable when long infusions are required. In this instance, the catheter body of FIG. 4 should be flexible and anchoring means (e.g., as discussed with respect to FIGS. 7-9) may be required. Removal from the retraction device may be accomplished in a number of fashions. One removal method involves pulling the retraction device over the catheter. In this instance, a removable fitting is required such that the catheter can be primed ahead of placement and then be hooked up again to the delivery means (infusion pump or syringe). Such removable devices typically are based on compression fittings.

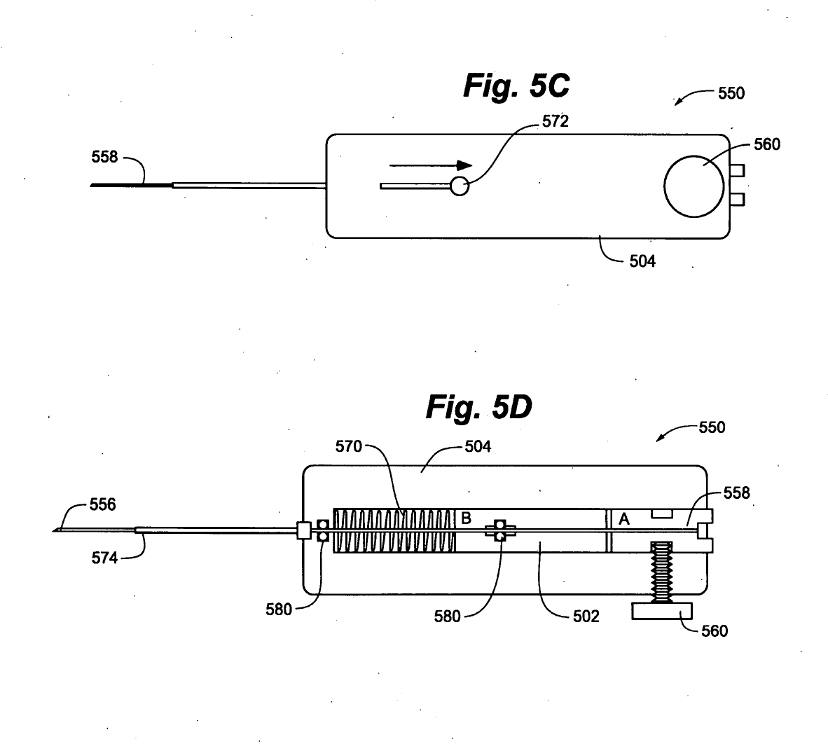

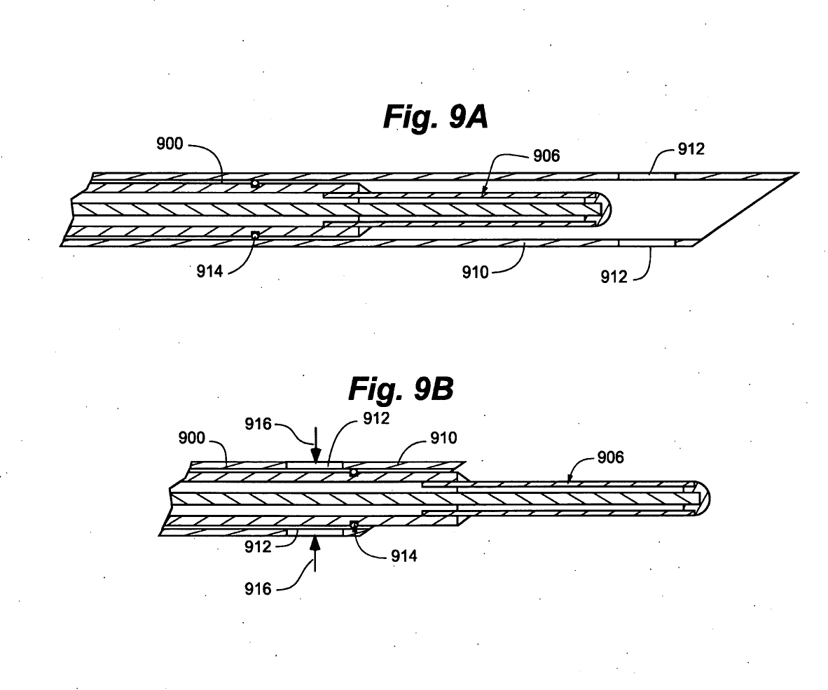

FIGS. 6A-6E provide cross sectional views illustrating an optional catheter priming method according to the current invention. In this example, a catheter 600 has two lumens to allow for air removal and priming. FIGS. 10-13 provide a more detailed description of one such catheter. Returning to FIGS. 6A-6E, the valve 602 is opened on the proximal end 604 of the catheter 600 and the infusate 606 is slowly injected through the center lumen 608. The infusate travels to the distal end 610 of the catheter and fills the hollow fiber 612. The hollow fiber 612 provides enough resistance to allow the infusate 606 to fill the remaining deadspace of the catheter 600 through a gap 614 created between the fiber 612 and support tube 616. The valve 602 is closed once the catheter is fully primed. A fluid pathway is now present through the hollow fiber 612. Accordingly, the illustrated example provides a user-friendly priming method that can greatly reduce the chance of introducing air into catheter 600. In the event that air bubbles are trapped in the fiber, they can be removed through the output lumen or gap. In addition, no hollow stylet is needed for placement of the catheter. In addition, although not shown, in some cases priming can also be accomplished with non-hollow fiber catheters, such as in conjunction with negative pressure.

A catheter of the present invention can be secured or anchored in the desired position using any suitable means, or corresponding mechanism. Anchoring can be desirable, for example, for long infusion periods. Suitable anchoring can be achieved, for instance, by the use of a coil or spiral wire positioned at the distal and/or proximal portions of the catheter, by means of suturing or other such means, by providing one or more hooks at the tip or other suitable portion (e.g., either associated with the hollow fiber portion or introducer needle), by the application of suction, by the use of surface features (e.g., texturing) sufficient to permit the catheter to be sufficiently retained in tissue, an expandable or deployable balloon (e.g., at tip or proximal end of the fiber), by means of an expandable stent surrounding fiber, by the use of spiral wire at the distal end (e.g., sufficient to permit the catheter to be effectively screwed into the tissue), and/or by the use of an anchor in the introducer needle (e.g., exterior to the hollow fiber catheter portion).

FIGS. 7A and 7B show cross sectional views of a preferred distal tip anchor mechanism according to an embodiment of the invention. In this embodiment the catheter 700 includes two spring-type anchors 702 that are attached (e.g., bonded, welded, and/or soldered) to the tip 704 of the hollow fiber 706 and inner stylet 708. In this case the anchors 702 are made of spring steel or a shape-memory alloy (e.g., nitinol) tubing cut and formed to the desired shape. As shown in FIG. 7A, the anchors 702 are retracted inside the needle 710 during insertion of the needle and catheter. Once the catheter 700 is positioned within the desired tissue, the needle 710 is retracted and the anchors 702 automatically deploy to secure the fiber 706 within the surrounding tissue. To remove the catheter 700, sufficient force is required to pull the anchors 702 from the tissue. Alternatively, the needle 710 can be re-extended over anchors 702 to disengage them from the surrounding tissue.

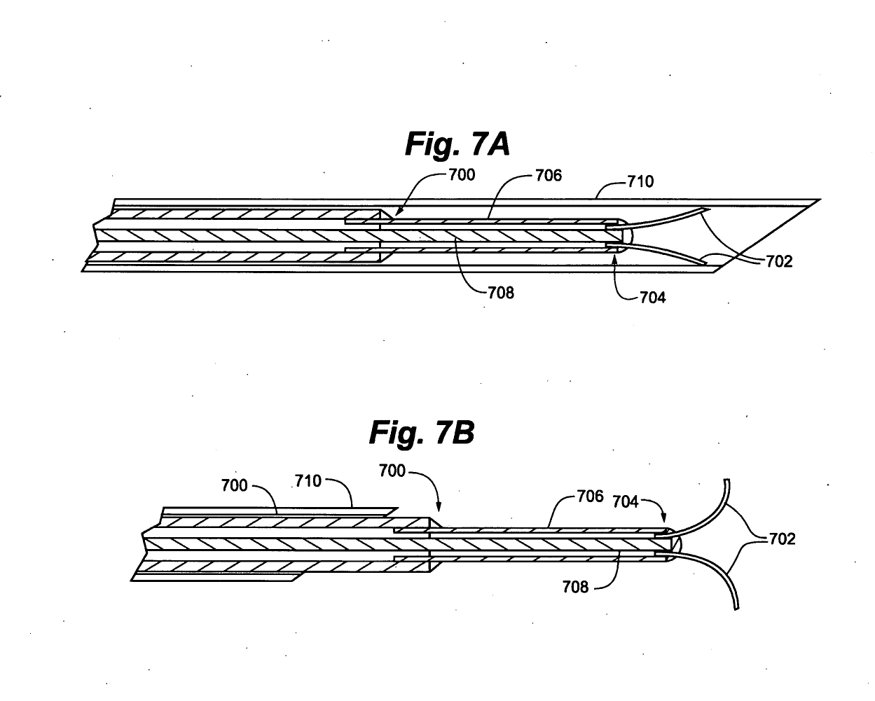

FIGS. 8A and 8B show cross sectional views of a preferred proximal anchor mechanism according to an embodiment of the invention. In this embodiment the catheter 800 includes two spring-type anchors 802 that are attached (e.g., bonded, welded, and/or soldered) at the proximal end 804 of the hollow fiber 806. In this case the anchors 802 are made of spring steel or a shape-memory alloy (e.g., nitinol) tubing cut and formed to the desired shape. As shown in FIG. 8A, the anchors 802 are retracted inside the needle 810 during insertion of the needle and catheter. Once the catheter 800 is positioned within the desired tissue, the needle 810 is retracted and the anchors 802 automatically deploy to secure the catheter 800 and fiber 806 within the surrounding tissue. To remove the catheter 800, sufficient force is required to pull the anchors 802 from the tissue. Alternatively, the needle 810 can be re-extended over anchors 802 to disengage them from the surrounding tissue.

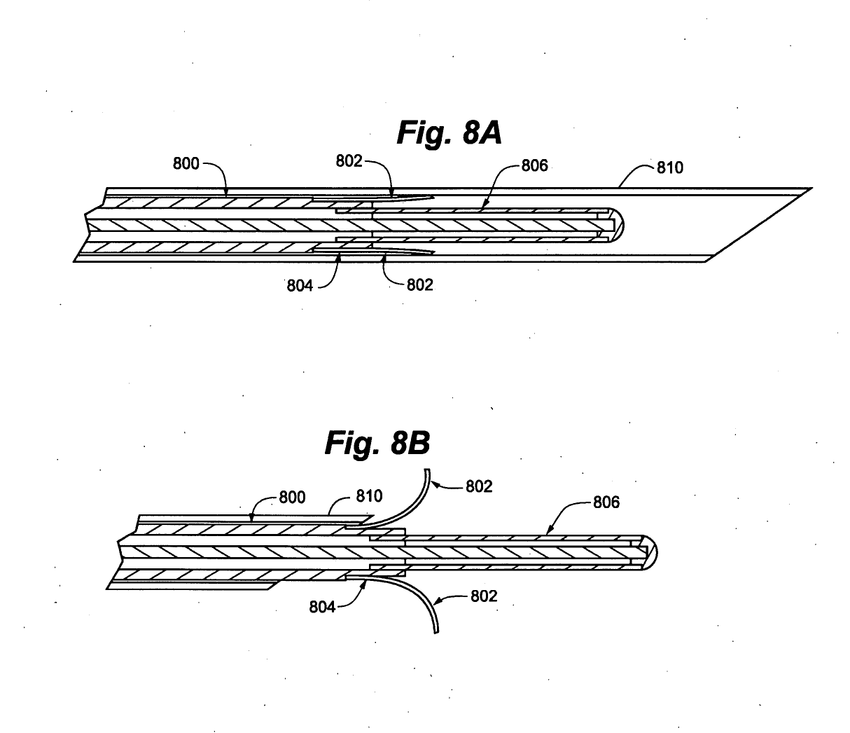

FIGS. 9A-9B show cross sectional views of a preferred mechanism for providing attachment through suction according to an embodiment of the invention. In this case the needle 910 includes one or more slots 912 at its distal end. After insertion, the needle 910 is pulled back to expose the hollow fiber 906 and position the slots 912 proximal to an o-ring seal 914. The o-ring seal is placed between the needle 910 and the catheter 900 to prevent suction distal to the needle slots 912. After retracting the needle 910 the desired amount, vacuum (suction) is applied to at the proximal end of the needle/catheter, creating a force 916 that pulls nearby tissue to the needle, thus securing the catheter 900.

In some cases, particularly where an internal stylet might be used in order to position the hollow fiber, once the stylet is removed, it tends to be difficult to then prime the hollow fiber, even under the application of positive pressure. In turn, in a particularly preferred embodiment, an apparatus of this invention provides an apparatus and corresponding priming method that is simple to use, and ideally does not require positive pressure (which can in some cases lead to inadvertent infusion). Such an apparatus can include, for instance, a plurality of lumen, including a first lumen for placement of the stylet, and one or more second lumen for the delivery of priming solution. As discussed above with reference to FIGS. 6A-6E, an optional catheter priming method can be performed using a catheter that has two lumens to allow for air removal and priming, either prior to or following placement. Accordingly, the catheter can be preferably positioned and used to avoid or minimize the formation of air bubbles within the hollow fiber, thus avoiding high infusion pressures and minimizing air displaced into the tissue.

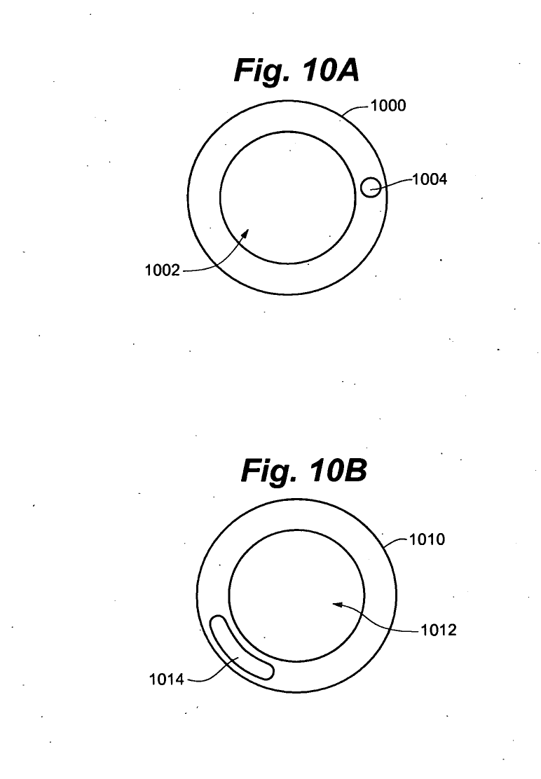

FIGS. 10A and 10B show cross sections of alternative embodiments of a catheter body having both a central (e.g., stylet) lumen and a priming lumen. The priming lumen can have any desirable cross-sectional shape. FIG. 10A shows one example in which the catheter body 1000 includes a stylet lumen 1002 and a priming lumen 1004 within the catheter wall having a circular cross-section. FIG. 10B shows one example in which the catheter body 1010 includes a stylet lumen 1012 and a priming lumen 1014 within the catheter wall having an extended or oblong cross-section. Turning to FIG. 11, the proximal end 1101 of a catheter 1100 includes fluid connections for both the central lumen 1102 and the priming lumen 1104, with luer locks 1106 providing connectivity, and showing also a leakproof enclosure 1108 that serves to seal the connection between priming lumen 1104 and the main body of catheter 1100. Optionally, and preferably, a valve (not show) or other suitable means can be provided in order to close the priming lumen 1104 once priming has been accomplished.

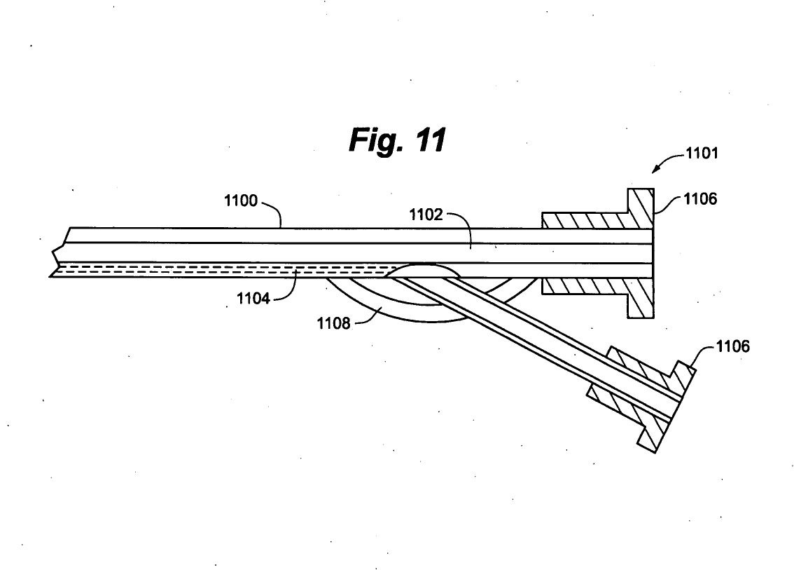

This priming method can be used with a variety of catheter types, of which two are shown in FIGS. 12 and 13. FIG. 12 illustrates the distal end of a standard end port catheter 1200 after the stylet has been removed. The distal end 1202 of the priming lumen 1204 is plugged and a hole 1206 near the tip of the catheter is created for fluid communication between the priming lumen 1204 and the stylet lumen 1208. In one particular example, infusate flow can follow the direction of the arrows, through the stylet lumen, through the hole, and back through the priming lumen.

FIG. 13 provides a cross sectional view of an infusion catheter 1300 incorporating a hollow fiber 1302 at the distal end to distribute the infusate. The hollow fiber 1302 is joined to the catheter body 1304 with a coupling and adhesive 1306. In addition, a support tube 1308 is positioned inside the hollow fiber 1302 and attached to the hollow fiber at the distal end with plug adhesive 1310. The support tube 1308 is also attached to the catheter body 1304 within the stylet lumen with an adhesive or other bond 1312. As with FIG. 12, the distal end of the priming lumen 1314 on the catheter body 1304 is plugged and a fluid communication hole 1316 is created. There are also one or more holes 1318 in the distal end of the support tube 1308 to allow for fluid communication from the inside the support tube to the hollow fiber and the priming lumen.

In practice, a fluid source, such as an IV bag or syringe, is attached to either the priming lumen or the stylet lumen, though IV bag or comparable is preferred since less vacuum is required to prime the catheter. A vacuum source is attached to the other lumen and applied with the valve open. Once fluid has reached the vacuum source, the valve can be closed, which is preferably attached to the fluid connection fitting attached to the vacuum source. Arrows in FIGS. 12 and 13 illustrate the priming direction for when the priming lumen fluid connection is the source of negative pressure/vacuum.

It is also possible to use positive pressure to prime the infusion catheter with the hollow fiber configuration as long as the priming pressure is kept below the fluid pressure required to push the fluid through the hollow fiber. In this instance a vacuum source is not required and a syringe can be attached to either fluid connection to prime the infusion catheter from the syringe with positive pressure and then closing the valve once fluid has reached the other fluid connection.

Returning to FIGS. 5A-5B and 5C-5D, it can be useful in certain embodiments to provide an infusion catheter with a retraction mechanism. FIGS. 14A-14C provide side and cross sectional views of a further preferred needle retraction device 1400 according to an embodiment of the current invention. The device 1400 includes of a spring-loaded inner shuttle 1402 used to retract the needle 1404 and expose the hollow fiber 1418 of the catheter 1410. First, the catheter 1410 (e.g., any of the catheters described herein, including the example of FIG. 13) is loaded and secured to the handle 1412. An index pin 1414 on the shuttle 1402 is pushed forward to compress the spring 1416 and allow the needle 1404 to cover the fiber 1418 of the catheter for insertion. Once the needle 1404 is placed into the tissue, the index pin 1414 is moved back along the handle 1412 and the needle 1404 retracts into an outer sheath 1420 attached to the handle 1412. The spring 1416 should have sufficient force to overcome tissue and o-ring resistance. The outer sheath 1420 helps with maintaining catheter position in tissue. Two o-rings 1422 are placed within the handle 1412 to provide air-tight seals and prevent fluid backflow up through the needle and the sheath. A luer fitting 1424 allows for easy fluid line connection.

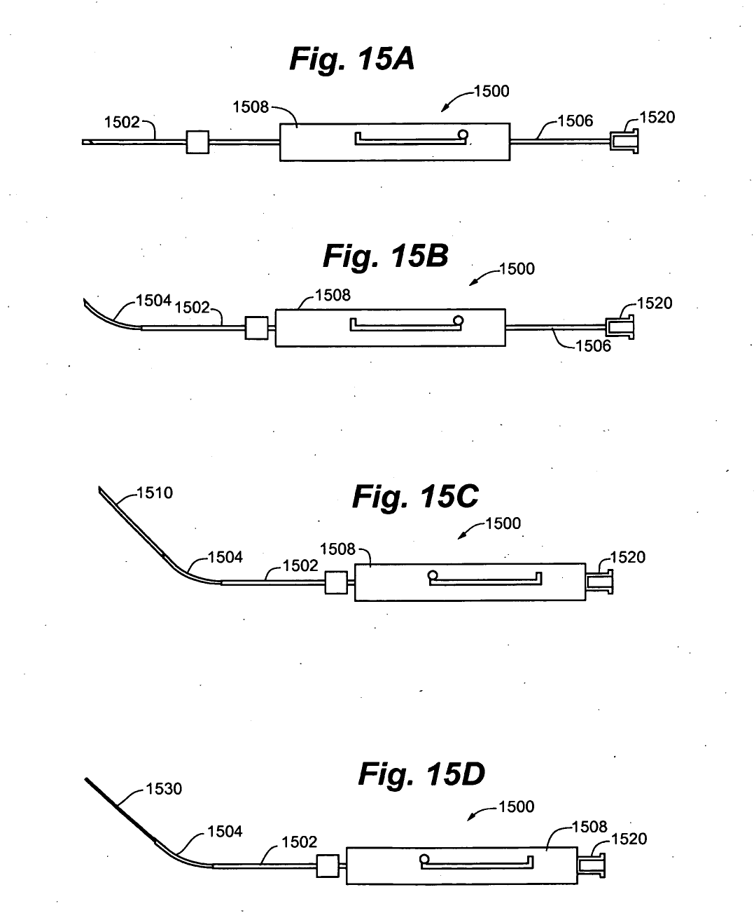

FIGS. 15A-15D provide views of an alternative needle retraction device 1500, including the use of a curved catheter tip. Such a device can be useful for delivery of a catheter into tissue requiring a curved needle (e.g., trans-urethral delivery into the prostate). Starting in FIG. 15A, an outer sheath 1502 can be extended over a curved needle 1504 as the catheter 1506 and needle are inserted through a scope. Turning to FIG. 15B, holding the outer sheath 1502 and simultaneously pushing the handle 1508 extends the curved needle 1504 out from within the sheath and into the surrounding tissue. FIG. 15C shows an additional straight needle 1510 (with catheter inside) extended through the curved needle 1504 into the tissue. Finally, as shown in FIG. 15D, the luer connection 1520 can be locked to the handle 1508 and the straight needle 1510 retracted to expose the distal tip of the catheter (e.g., and hollow fiber 1530).

The apparatus and method of the present invention can be used to provide delivery in a manner that meets or exceeds those required for convective flow, and in turn, significant mass transfer. The velocity of convective flow tends to be the key energy-consuming variable, and the flow velocity density, as defined by Darcy's Law, is related to hydraulic conductance. Hydraulic conductance, in turn, depends on the size of the extracellular space: the greater the extracellular space, the higher the cross-sectional area available for flow, and the lower the flow velocity for equal volumetric flow rates. Thus, if all other factors are equal, flow velocity is the limiting, energy-consuming aspect of interstitial fluid movement, and therefore a key parameter to consider for drug delivery. Further, at the level of the interstitial space, the relationship of mass transfer coefficients, contact time, and diffusion coefficients (Sherwood number) means that mass transfer may be optimal at minimal flow velocity. Maintenance, or induction, of increased extracellular space volume is an important goal that may be accomplished with hollow fiber technology (HFT) according to the method and apparatus of the present invention.

As compared to the direct interstitial infusion of bioactive agent using conventional needles, the apparatus and method of the present invention have the potential to provide various improvements, including with respect to distribution pattern, flow rate, and backflow.

The velocity of fluid movement is the energy-consuming, rate-limiting step of convectively moving a drug through the tissue. For instance, Rosenberg et al. (1980) determined the velocity of fluid movement in white matter to be 10 μl/min toward the ventricle of a normal brain. By comparison, Bauman et al. (2004) calculated the velocity of nanoscale flow in isotropic tissue phantoms to be 10 microns/sec. If flow into the tissue (by infusion) exceeds the capacity of flow within the tissue, impedance mismatch can occur. Convective flow within the tissue is determined by surface area times velocity of flow times available extracellular volume.

Given the limitations of shearing and backflow associated with conventional CED, infusion rates are generally limited to 0.1-0.5 μL/min using a 32-gauge needle. (Morrison et al. 1999) Using very slow infusion, Bobo et al. (1994) documented the benefit of convection-enhanced interstitial infusion in the brain. They used a single-delivery catheter and infused artificial cerebral spinal fluid at up to 0.4 μl/min. No attempt was made to remove excess fluid, as they relied on natural tissue drainage mechanisms. Slow infusion resulted in a homogenous distribution over the hemisphere.

Those skilled in the art, given the present description, will be able to determine the appropriate delivery parameters needed in order to achieve whatever flow and distribution might be desired, on a patient by patient basis.

The apparatus and method of this invention can be used for injection into any suitable organs and tissue, such as prostate muscle, liver, breast, and lungs, and is particularly preferred for use with tissue sites that might otherwise encounter access or distribution problems associated with conventional local delivery.

In a preferred embodiment, for example, the apparatus and method of this invention addresses difficulties previously encountered in the course of injection into the prostate in order to treat BPH, given the complex structure of the cone-shaped glandular lobules. There are approximately 20-70 tuboalveolar glands in humans, converging into 16-32 ducts that drain into the prostatic urethra. Given the solid and ductal components of the prostate, direct injection into the prostate presents unique challenges. An apparatus of this invention can be used to deliver the therapeutic agent into the solid component as well as the acinar component, and can do so across multiple glandular lobules.

In turn, one problem that is associated with conventional needle injection into the prostate, is that all the injectate is delivered to a single point; if that point happens to be within an acinar, distribution will be excellent within the lobule, particularly within the acinar volume. However, there will be very little injectate crossing into the solid component or through the fibromuscular layer into adjacent lobules. If the needle tip is within a vessel, a significant component of the injectate may be absorbed systemically. The apparatus and method of this invention can be used to address and overcome these and other obstacles involved in the delivery of drugs to the prostate, and in turn, provides a critical step in attaining better clinical outcomes.

Conventional concerns regarding backflow and irregular distribution can be improved using a present apparatus for prostate injection as well. The agents that are most effective in shrinking the prostate are caustic agents, and backflow of injectate out of the prostate can cause significant injury to important surrounding structures such as the rectum and the urethra. Necrosis of these and nearby structures can cause significant lifelong morbidity. Irregular distribution is less of a safety issue, but correct dosing becomes difficult. Insufficient treatment tends to require repeated procedures, in order to achieve a clinically significant relief in lower urinary tract symptoms and quality of life.

Direct infusion of drugs into the prostate using convection-enhanced delivery (CED), using the method and apparatus of the present invention, can result in the treatment of large areas of tissue, concentrating the infusate in situ. CED is a technique that relies on bulk flow to establish a pressure gradient, resulting in continuous convective flow and widespread distribution of the infusate in the tissue. The extent of drug distribution achieved using CED depends on many factors, including the following: hydraulic conductance of the tissue, interstitial pressure, type of tissue infused, molecular weight of infusate, volume and flow rate during administration, and diameter/type of administration catheter(s). The prostate presents unique challenges in that the injectate must be delivered across a wide area to both solid and acinar components.

The present invention addresses concerns associated with conventional CED, for instance, the problems that can result from delivery of infusate at a rate greater than the capacity of the tissues to take up the fluid, for a given needle/tissue area of interface. A delivery method that increases the volume of distribution while maintaining clinically relevant infusion rates, such as that presently described, can provide a significant advancement for the field.

Models have been studied that involve the analysis of interstitial flow and impedance mismatch. Under conditions of idealized flow, every cell is perfused with convective fluid flow. In the event the infusion rate is increased, an impedance mismatch exists, and all the fluid delivered by the catheter does not flow into the tissue. The resultant increase in pressure at the fluid-tissue interface creates a force that tends to deform the tissue, which in turn decreases the size of the interstitial pathways nearest the catheter, and further increases resistance to tissue flow. With continued infusion, rising pressure can create a rupture in the tissue. Any therapeutic agent in this interstitial pool can then only reach the cells by diffusion.

Given these and other considerations, hollow fiber catheters can be used to address and prevent asymmetric or other irregular distribution, as well as the concerns arising from backflow, that tend to be seen in conventional prostate injections.

Those skilled in the art will appreciate the manner in which hollow fibers can be selected and used in the manner presently described in order to provide various properties, including:

1. Low axial over radial resistance ratio

2. Dispersed pressure fields

3. Tissue interaction
a. Extended surface area
b. Limited infusion force
c. Pore connectedness

4. Clinical applicability and safety
a. Small outside diameter
b. High rupture strength
c. Biocompatible materials

5. High transmittance rate of large molecules

The factors (1) and (2) are particularly advantageous for delivery of drug into the glandular structure of the prostate, while factor (3) can be of importance for use in the solid tissue of the prostate. The factor (4) is an indication of early clinical approval for the proposed therapy. Factor (5) means that large molecules can be delivered by the hollow fiber catheters.

With regard to the feature involving low axial over radial resistance ratio, hollow fiber catheters can produce uniform delivery along the length of the catheter, due in large part to both their high transmural resistance and corresponding low intraluminal resistance to flow. In turn, and as compared to multiple-pore catheters, transmural outflow is reasonably heterogeneous from proximal to distal. Hollow fibers have been shown to provide reproducible, cylindrical distribution of the infused substance into test systems such as agarose gel. Hollow fiber catheters have been tested in skeletal muscle as well, and produced similar cylindrical distribution.

The term “dispersed pressure fields” refers to the situation in which infusion pressure can be relatively evenly dispersed across the surface of the hollow fiber, in contrast to a needle, where all the pressure and flow is at the needle tip. Because of the high transmural resistance to flow, outflow occurs throughout the length of the hollow fiber, even though a portion of the fiber may be in a low resistance area. Only slightly more drug may be delivered into low pressure areas compared to higher pressure areas. In the course of evaluating for this property, a tissue phantom is prepared with a gap in the gel near the midpoint of the hollow fiber. A suitable dye is infused at 10 through 60 minutes of infusion. While dye does fill the space (analogous to ductal elements in the gland), the solid portion of the tissue phantom is also infused, and again with a cylindrical distribution. This concept was tested in a tissue phantom of 0.6% agarose gel, a model that has been firmly established for CED in human brain. Prior to pouring of the gel, a 3-mm Teflon sheet was placed around the hollow fiber prior to gelation, and the sheet was removed before dye infusion. An infusion pump (KD Scientific) was used to infuse 0.1% Evans Blue dye into the gel at a constant rate of 5 μL/min for 2 hours. In turn, it could be seen that even with the gap, there was dye delivery to the gel both proximal and distal to the gap. This, is an important safety factor provided by the hollow fibers as compared to needles, in which the entire dose is delivered at the tip of the needle. If the needle tip happens to be in an artery, vein, duct, or tissue plane, distribution will be affected. By contrast, a hollow fiber catheter traversing these structures will be minimally affected. For prostate infusion, this characteristic of hollow fibers means that both acinar elements and solid elements of the glandular lobules will be infused with therapeutic agent.

In terms of tissue interaction, and particularly extended surface area, flow is a product of velocity and cross-sectional area. Thus, a large transfer plane will produce large flow rates. For instance, considering the surface area of the open tip of a 27-gauge needle, a 4-cm hollow fiber catheter of the same diameter provides 271 times the surface area. Similarly, with regard to pore connectedness, the porosity of the hollow fiber catheters essentially replicates the porosity of tissue, so “impedance mismatch” can be minimized or entirely avoided. Given the substantially homogenous interstitial flow that can be achieved with an apparatus of the present invention, distribution to every cell can be enhanced. In turn, the advantages of pore connectedness within the tissue interstitial space can be utilized for drug delivery.

In terms of limited infusion force, yet another advantage of hollow fiber catheters involves the relatively small pores these fibers provide. Application of infusion pressure, using conventional needles, will create force on the tissue, which in turn can deform tissue, particularly the extracellular space, and will result in increased resistance to convective flow. Because force is a product of pressure and area, the small pore size of the present invention tends to limit if not entirely avoid tissue deformation.

A comparison was made as between needle infusion and human scale hollow fiber catheters. To demonstrate improved tissue distribution and less shearing and backflow, 2-cm hollow fiber catheters were compared to needle infusion in a 0.6% agarose gel tissue phantom. The hollow fiber catheter exhibited better infusion characteristics than the conventional needle. In the five trials, four of five needles resulted in reflux or shear planes, while none of the hollow fiber catheters resulted in reflux or shear planes (significant at p<0.05 by Fischer Exact test).

Hollow fibers suitable for use in this invention are typically clinically applicable and safe for such purposes. In particular, smaller catheter widths tend to limit tissue trauma during insertion, yet still provide a relatively large surface area. A typical hollow fiber catheter is about the size of a 27 gauge needle. Suitable fibers also provide sufficiently high rupture strength, in that they can tolerate high pressures before rupture. Nominal bursting strength of a preferred hollow fiber is about 25 pounds per square inch or greater.

Preferred hollow fibers can have any suitable pore size, e.g., 45-μm pore size, which, theoretically, will allow passage of up to 1,000,000 Da. Because most therapeutic agents can pass through a 0.2-μm IV fluid filter used in pharmacies, such agents are likely to be transmitted using the apparatus and method of the present invention. Extensive studies have been performed to demonstrate high transmittance of small agents such as dyes, chemotherapeutics, and antibiotics. Combination of dye and iohexal together were easily transmitted through the hollow fiber.

In order to achieve success in humans, a preferred hollow fiber catheter of the present invention should meet three general requirements in a timely and reliable fashion, namely: (1) ability to be positioned and retained at the targeted location, (2) widespread infusate distribution within the tissue site (e.g., prostate); and (3) removal without complications. There is sufficient clinical experience with needles in prostates, used in conjunction with transrectal ultrasound (TRUS), to demonstrate that meeting the placement and removal performance of prostate needles will be adequate for prostate hollow fiber drug catheters. Thus, equivalent placement and removal performance for the hollow fiber catheter as compared to standard of care needles will meet this requirement. Since placement of the hollow fiber catheter will be accomplished in conjunction with the same needle gauge as used for injections with transrectal ultrasound (TRUS) needle, catheter placement is expected to be equivalent to standard of care prostate needles.

An infusion catheter of this invention comprises at least one, and optionally a plurality, of semipermeable membranes. As used herein, the term “semipermeable membrane” will generally refer to a membrane forming some or all of the wall of a microcatheter (e.g., “hollow fiber”), preferably with a substantially open lumen having at least one open end accessible to liquid or fluid flow within the lumen. The membrane portion itself is adapted to permit the passage of bioactive agent, while substantially precluding the passage of cells or non-fluid tissue. Such passage can be accomplished using any suitable means, e.g., through pores provided by the membrane itself, as well as by the preparation of membranes having suitable chemico-physical properties (e.g., hydrophilicity or hydrophobicity) to effectively control passage of fluid and its components in a predictable and desired fashion.

An introducing components, in turn, can include any introducing component, or set of components, that is suitable and adapted to position the recovery catheter(s) within a tissue site, and preferably within a tissue site. Such components can be provided, for instance, in the form of a totally or partially circumferential covering (e.g., stationary or removable delivery sheath), and/or by the inclusion of one or more components (e.g., stylets) positioned internally, adjacent to, and/or along the length of the semipermeable membrane(s) and designed to impart sufficient properties (e.g., stiffness, lubricity) to the overall catheter assembly or portions thereof.

The catheter(s) can be provided in any suitable form and configuration, e.g., as one or more closed and/or open ended individual fibers, as a plurality of closed and/or open ended parallel fibers, and/or as circuitous loops of fibers. In such configurations, the lumen of each catheter will typically include an entry orifice for the delivery of infusate.

The fibers can be delivered to the tissue site using any suitable introducing components, e.g., they can be positioned within a surrounding placement catheter (e.g., conventional catheter or customized introducer) that can itself be removed or permitted to remain in place in the course of using the delivery/recovery catheter. Optionally, or in addition, the delivery/recovery catheters can be accompanied by one or more delivery guidewires, stylets, or trocars, and combinations thereof, e.g., adapted to position the semipermeable membrane(s) within the tissue site.

The length (l) of the fibers can be on the order of about 3 mm to about 100 cm and preferably is between about 1 cm and about 10 cm. The radius (r) is typically derived using fibers having an inner diameter (ID) of between about 50 microns to 5000 microns, and more preferably about 100 microns to about 1000 microns.

Suitable monitors include, but are not limited to, those adapted to qualitatively and/or quantitatively assess various parameters, preferably in a substantially “real time” fashion during and in the course of using a system of this invention. Such parameters can include physiologic parameters associated with the tissue itself, as well as performance parameters associated with the function of the system or its components. Examples of suitable physiologic parameters include, but are not limited to, tissue pressure (total and partial pressures), blood flow, hydration (water content), temperature, pH, sodium, and biochemical parameters (e.g., myoglobin levels).

Such parameters can be determined using any suitable means, for instance, pressure can be determined using conventional fluid column techniques (e.g., diaphragm or manometer), or fiberoptic techniques, while fluid (including blood) flow can be determined using near IR spectroscopy and laser Doppler techniques, and tissue hydration can be determined by a variety of means, including the placement of a suitable probe or electrode to determine electrical impedance.

Suitable materials for use as semipermeable membranes of the present invention provide an optimal combination of such properties as mass transfer properties, biocompatibility, surface-to-volume ratio, processability, hydrophobicity and hydrophilicity, strength, transport rate, and porosity. Examples of suitable hollow fibers are described, for instance, I. Cabasso, “Hollow-Fiber Membranes”, pp 598-599 in Kirk Othmer Concise Encyclopedia of Chemical Technology. In a preferred embodiment, such membranes are provided in the form of “hollow fibers” or “microcatheters”, having walls (or portions thereof) formed of such membrane material. In alternative embodiments, the membranes can be provided in any suitable form or configuration, e.g., in the form of pleated or corrugated membrane sheets, and the like, preferably positioned within and/or by a recovery catheter. In situations where the semipermeable membranes) are provided in other than circumferential (e.g., fiber) form, the hydratable medium can be delivered to a major surface of the membrane, opposite the surface in contact with, or accessible by, the tissue fluid itself.

The dimensions of a hollow fiber will depend largely on the intended use of the apparatus. In a number of preferred embodiments, a hollow fiber will be provided in the form of a capillary having an outer diameter of between about 0.1 mm and about 10 mm, preferably between about 0.2 mm and about 3 mm, and more preferably between about 0.3 mm and about 1 mm. Such capillary fibers preferably also provide a substantially open lumen, defined by an inner fiber diameter that is typically on the order of 50% or more, and preferably 70% or more the corresponding outer diameter.

Such membranes preferably also provide permeability cutoffs suitable for use in the intended application. The permeability of hollow fiber membranes for use as microdialysis fibers is generally phrased in terms of kilodaltons (and can range between about 10 kD to about 1000 kd). By comparison, the permeability of fibers used for ultrafiltration is typically considerably greater, and hence phrased in terms of microns, with typical ranges from about 0.1 micron (corresponding roughly to the 1000 kD cutoff at the higher range above) to about 1 micron. Fibers suitable for use in the system of the present invention, therefore, typically provide permeability in the range of from about 1 kD to about 200 microns, preferably from about 10 kD to about 10 microns, and more preferably between about 50 kD and about one micron.

Permeability can be determined using suitable techniques, such as conventional wet sieving techniques. See, for instance, Spectrum Laboratories, Inc. product information which describes the manner in which both the membrane molecular weight cut-off (MWCO) and pore size are related and can be determined.

Optionally, and preferably, microcatheters used in this invention can have regions of varying characteristics, including varying porosity, rigidity, and the like, for instance those that vary between sequential and adjacent, or suitably spaced, longitudinal sections, or in or any other suitable pattern. Such variations can be used, for instance, in a size exclusion fashion to improve or provide the ability to retain or permit the passage of solutes of varying sizes in a predetermined manner. Such variations can also be used to provide regions of greater rigidity or varying structure (e.g., fluted), in order facilitate their placement in tissue. Such variations can also include the incorporation of means (e.g., radioopaque materials) to facilitate the visualization of implanted catheters. Such variations can also be used to place regions of semipermeable membranes in desired locations within the tissue, e.g., in order to effect a gradient between two or more regions, or to avoid the placement of semipermeable regions in particular tissues or areas thereof.

In turn, the present invention provides a hollow fiber catheter system for use in delivering fluids in a controlled fashion to the body, and in particular, for infusing fluids into the body, the system providing an improved and optimal combination of properties, including controlled deliver of fluids containing bioactive agents, while minimizing shear plane and reflux, as well as backflow and asymmetric delivery of an agent. A system as described herein, can include a needle retraction mechanism that allows for one handed operation, and optionally, including a mechanism to provide the force for the retraction of the needle. A system can also include a mechanism for use in anchoring to tissue. A system as described herein, can be adapted for use in infusing into organs and tissue such as prostate muscle, liver, breast, lung and other that encounter similar problems for controlled local delivery.

A study of drug distribution in a canine prostate model has been completed. Microporous catheters and needles (single end port) of the same diameter were compared in eight dogs. Ethanol was infused to reduce prostate size. The prostates were harvested and examined histologically for necrosis due to ethanol. Each prostate had a needle and hollow fiber injection on separate sides of the prostate using the same infusion flow rates and volumes. The necrosis analysis indicated the microporous catheter produced a 64% increase in drug distribution compared to needles and was significantly different by paired t test (p=0.001).

To study the effect of hollow fiber delivery of large molecules (>50 kDa), we studied the efficiency of gene transfer using a recombinant adenovirus (˜80 nm in diameter; >120 kDa) that encodes the firefly luciferase enzyme. The hollow fiber-mediated gene transfer and expression was over one log higher than when a normal needle was used to deliver the identical dose/volume. While infusing tobramycin ((1,425 Da) at 1 mL/min, infusion pressure did not exceed 500 mmHg, which is another indication that large molecules will not occlude the pores of the hollow fiber over time. Transmittance of carboplatin has also been verified by bioassay (a live cell culture). Proteins as large as 250 kDa have been recovered from human tumors.

Hollow fiber catheters have been used to infuse Evan's Blue dye into ex vivo canine prostates. After several pilot studies, this study objective was to evaluate dye distribution at a clinically desirable injection time of 10 minutes or less. A 24 ml canine prostate was harvested. A balloon catheter was inserted into the canine urethra and partially inflated to prevent dye flowing into the urethra from the prostatic ducts. The prostate was encapsulated in 0.6% agarose gel for stabilization. A Tuohy Borst was connected to a 21 gauge needle to facilitate hollow needle insertion and retraction. Two 1.4-cm hollow fiber catheters were primed with 0.01% Evan's blue dye in saline, and positioned in the needles, which are then both inserted into the lobes of the prostate. The proximal ends of each catheter were secured and Tuohy Borst loosened. Slowly, the needle was pulled back to expose the hollow fiber. Infusion pressure was monitored. After infusion, the prostate was cooled for 1 hour and then 5 mm slices are prepared. The needle and catheter device was inserted with ease into each lobe. The pressure reached 401 and 480 mm Hg at 10 minutes for the left and right lobe infusion respectively. Total volume infused was 3.6 milliliters per catheter. No backflow was observed with either hollow fiber catheter. Evan's blue dye was distributed in each lobe with very little dye traveling into the urethra.

What is claimed is:

1. An apparatus for infusing a tissue site, which comprises:
a) one or more hollow fiber catheters adapted to be placed and retained in a desired position within a tissue site, and following use removed from the tissue, without undue damage to the tissue;
b) a bioactive agent circuit adapted to deliver bioactive agent from a source and to the catheter(s) in order to be delivered to the tissue site; and
c) a control mechanism adapted to permit the controlled delivery of bioactive agent to the catheter(s) in a predetermined manner to the tissue site.

2. The apparatus of claim 1 wherein the one or more hollow fiber catheters are adapted to be placed into and positioned within the tissue site using ancillary means.

3. The apparatus of claim 2 wherein the ancillary means are selected from the group consisting of a removable sheath, trocar and/or stylet that provides suitable properties to the catheter in the course of its delivery.

4. The apparatus of claim 3 wherein the properties are selected from the group consisting of strength, rigidity, the ability to be steered or otherwise positioned, and the ability to be tracked or located by suitable means.

5. The apparatus of claim 4 wherein the one or more catheters are adapted to be substantially retained in position within the tissue site during the course of infusion.

6. The apparatus of claim 1 wherein the catheter(s) comprise distal and/or proximal anchor mechanisms.

7. The apparatus of claim 1 wherein the bioactive agent circuit is adapted to deliver bioactive agent substantially without the presence of air or other occlusions.

8. The apparatus of claim 1 wherein the apparatus comprises means for first priming the catheter with a suitable solution.

9. The apparatus of claim 1 wherein the control mechanism permits one handed operation of the apparatus and/or one or more delivery controls.

10. The apparatus of claim 9 wherein the delivery controls are selected from the group consisting of control of position, flow rate, timing, and corresponding detectors.

11. A method for infusing a tissue site with bioactive agent, the method comprising: a) providing an apparatus according to any previous claim,
b) placing and retaining the catheter(s) within a tissue site, in a manner that positions the hollow fiber portion in an desired position and orientation with respect to the tissue,
c) activating the apparatus in order to deliver bioactive agent to the tissue in a manner that provides an optimal and predetermined combination of properties selected from the group consisting of delivery kinetics and distribution.

12. A method according to claim 11, wherein the one or more hollow fiber catheters placed into and positioned within the tissue site using ancillary means selected from the group consisting of a removable sheath, trocar and/or stylet that provides suitable properties to the catheter in the course of its delivery.

13. A method according to claim 12 wherein the properties are selected from the group consisting of strength, rigidity, the ability to be steered or otherwise positioned, and the ability to be tracked or located by suitable means.

14. A method according to claim 13 wherein the one or more catheters are substantially retained in position within the tissue site during the course of infusion.

15. A method according to claim 14 wherein the one or more catheter(s) are retained in position by the use of distal and/or proximal anchor mechanisms.

16. A method according to claim 15 wherein the bioactive agent is delivered substantially without the presence of air or other occlusions.

17. A method according to claim 16 wherein the one or more catheters are first primed with a suitable solution.

18. A method for infusing a tissue site with bioactive agent, the method comprising: a) providing an apparatus that comprises
i) one or more hollow fiber catheters adapted to be placed and retained in a desired position within a tissue site, and following use removed from the tissue, without undue damage to the tissue;
ii) a bioactive agent circuit adapted to deliver bioactive agent from a source and to the catheter(s) in order to be delivered to the tissue site; and
iii) a control mechanism adapted to permit the controlled delivery of bioactive agent to the catheter(s) in a predetermined manner to the tissue site,
b) placing and retaining the catheter(s) within a tissue site, in a manner that positions the hollow fiber portion in an desired position and orientation with respect to the tissue, by the use of one or more anchors associated with the one or more catheters, respectively,
c) priming the catheter with solution prior to, during, or following placement in the tissue site, and activating the apparatus in order to deliver bioactive agent to the tissue in a manner that provides an optimal and predetermined combination of properties selected from the group consisting of delivery kinetics and distribution.
